(19) United States
(12) Patent Application Publication
(10) Pub. No.: US 2012/0129764 A1
(43) Pub. Date: May 24, 2012

(54) INFLUENZA A AND B VIRUS REPLICATION-INHIBITING PEPTIDES

(75) Inventors: Ulrich Kessler, Boppelsen (CH); Daniel Mayer, Zurich (CH); Kerstin Wunderlich, Leiden (NL); Charlene Rnadheera, Winnipeg (CA); Martin Schwemmle, Freiburg (DE)

(73) Assignee: PIKE PHARMA GMBH, Zurich (CH)

(21) Appl. No.: 13/318,769

(22) PCT Filed: May 8, 2009
(86) PCT No.: PCT/EP09/55632

§ 371 Date: Jan. 26, 2012

(51) Int. Cl. A61K 38/10; C07K 7/06; G01N 33/53; A61K 38/08; A61P 31/16; C07H 21/04; C07K 14/00; A61K 38/16; C07K 7/08; G01N 21/75

(52) U.S. Cl. 514/3.7; 530/326; 530/327; 435/7.92; 436/86; 536/23.1; 530/324; 530/328; 530/329

(57) ABSTRACT

A synthesized or isolated influenza virus replication-inhibiting peptide that competitively inhibits protein-protein interaction of the PA and PB1 of both influenza Virus Types A and B and novel in vitro binding screen to identify peptides with antiviral activity against influenza viruses of both type A and B is disclosed. In addition to the well-known pandemic influenza A viruses (such as the 1918 “Spanish” flu or H5N1), both type A and B viruses contribute greatly to the annual recurring epidemics that cause the vast majority of human cases and medical cost. Surprisingly, it was found that the novel virus replication-inhibiting, are able to inhibit protein-protein interaction of the PA and PB1 subunits of the heterotrimeric viral RNA polymerase complex of both influenza virus types A and B. The viral polymerase sub-unit interaction domain turned out as an effective target for the new antivirals, as correct assembly of the three viral polymerase subunits PB1, PB2 and PA is required for viral RNA synthesis and infectivity.

### REFERENCE TO RELATED APPLICATIONS This application is the US national phase entry of International Patent Application No. PCT/EP2009/055632, filed May 8, 2009.

### FIELD OF THE INVENTION

The present invention relates to influenza virus replication-inhibiting peptides which inhibit influenza A and B virus replication; influenza virus-replication inhibitors which inhibit influenza virus replication; methods for determining influenza polymerase subunit interaction inhibitors and influenza therapeutic agents comprising an influenza virus replication-inhibiting peptide.

### BACKGROUND OF THE INVENTION

Influenza viruses are negative-stranded RNA viruses that cause yearly epidemics as well as recurring pandemics, resulting in high numbers of human cases and severe economic burden. In addition to the well-known pandemic influenza A viruses (such as the 1918 “Spanish” flu or H5N1), both type A and B viruses contribute greatly to the annual recurring epidemics that cause the vast majority of human cases and medical cost. The WHO recommends an annual vaccination against circulating influenza A (FluA) and B (FluB) strains. However, current vaccines confer incomplete protection against epidemic influenza. To date, only the neuraminidase inhibitors oseltamivir (Tamiflu) and zanamivir (Relenza) are available as antiviral treatment against both virus types. However, there is a growing fear within the medical community about the rapidly growing emergence of influenza strains resistant to both drugs. The older adamantane drugs are not effective against FluB and the global spread of influenza viruses resistant to oseltamivir demonstrate the limitations of this class of drugs. A recent epidemiological survey in the U.S. found 98.5% of the H1N1 isolates tested resistant to oseltamivir. Thus, new improved and alternative antiviral agents against both virus types are urgently needed.

### SUMMARY OF THE INVENTION

It is an object of the present invention to provide new, improved and/or alternative influenza antiviral agents.

It is a further object of the invention to obviate or mitigate at least one disadvantage of influenza antiviral agents known from the state of the art.

It is yet a further object of the invention to provide new improved and alternative methods for determining influenza polymerase subunit interaction inhibitors.

### DISCLOSURE OF THE INVENTION

Surprisingly, it was found that the novel virus replication-inhibiting peptides in accordance with the present invention, are able to inhibit protein-protein interaction of the PA and PB1 subunits of the heterotrimeric viral RNA polymerase complex of both influenza virus types A and B. The viral polymerase subunit interaction domain turned out as an effective target for the new antivirals, since correct assembly of the three viral polymerase subunits PB1, PB2 and PA is required for viral RNA synthesis and infectivity. Structural data for the entire trimeric complex is missing. Based on the crystal structure of a truncated FluA PA in complex with the N-terminus of PB1 it was established by the inventors that the crucial PA interaction domain of PB1 consists of a 3₁₀-helix formed by amino acids (aa 5-11). The domain is highly conserved and virus type specific among both, influenza A and B viruses (FIG. 1a).

Novel peptides according to the present invention, containing amino acid sequences from both virus types A and B, bind to PA subunits of both types of influenza A and B. Among said novel peptides, chimeric peptides, containing amino acid sequences from both virus types A and B, were identified which not only bind to both PA subunits, but also decrease the viral polymerase activity and the spread of virus in cell culture for both influenza A and B.

In one aspect, the present invention provides an isolated influenza virus replication-inhibiting peptide which has been shown to effectively interfere with the protein-protein interaction domains of PA and PB1 subunits of the heterotrimeric viral RNA polymerase complex and thereby causes inhibition of virus replication.

According to a further aspect of the invention an ELISA-based screening method to identify variant peptides, derived from the PA-binding domain of the PB1 subunit of the heterotrimeric viral RNA polymerase complex, which can bind to the PA subunit of both influenza A and B viruses is provided.

The present inventions makes it feasible to use the inventive peptides, together with the new ELISA-based screening assay to identify small molecule lead compounds which are antivirally active against influenza A and B viruses. Since such small molecules are effective against both virus types, they represent an attractive alternative to neuraminidase inhibitors and constitute a major step toward a sorely needed, near-universal pharmaceutical against influenza virus, and one which, due to its protein-protein interaction domain target, is likely be less susceptible to the emergence of drug-resistant strains for which influenza is well known.

The peptides according to the present invention comprise an amino acid sequence being at least 60%, preferably at least 70%, more preferably at least 80% or 90% identical to the polypeptide according to the wild type PB1₁₋₁₁A which is MDVNPTLLFLK (SEQ ID NO: 1). One or several amino acid residues may be substituted, deleted, or added and the protein has still inhibitory activity against protein-protein interaction of the PA and PB1 subunits of both influenza virus types A and B. However the already known wild type PB1₁₋₁₁A is explicitly disclaimed. The inventive peptides are synthesized or isolated influenza virus replication-inhibiting peptides that competitively inhibit protein-protein interaction of the PA and PB1 subunits of the heterotrimeric viral RNA polymerase complex of both influenza Virus Types A and B. Those peptides comprise an amino acid sequence, comprising the sequence of X₅X₆X₇X₈X₉X₁₀, wherein X₅ is P; X₆ is T, Y, F, W, H, C, I, L, V, A or M; X₇ is L or F; X₈ is L, I, F or M; X₉ is F, Y, W, H, L, R or S; and X₁₀ is L, I or Y (SEQ ID NO: 2).

According to preferred embodiments of the present invention the amino acid sequence of the inventive peptide is least 66%, preferably at least 73%, more preferably at least 79%, 86% or 93% identical to the polypeptide according to the wild type PB1₁₋₁₅A which is MDVNPTLLFLKVPAQ (SEQ ID NO: 3). The wild type per se is again disclaimed.

It should be noted that all amino acids are preferably indicated by the IUPAC one letter code in the present application. Whenever three letter codes are used, they are also in accordance with IUPAC. The letter X is used to indicate a wildcard/variable or other amino acid at a certain position.

According to a further aspect of the present invention an influenza virus replication inhibitor comprises at least one of said above described peptides fused to a cell-penetrating peptide, preferably a cell-penetrating domain of HIV-Tat, as an active ingredient and inhibits replication of influenza A and influenza B strains. According to further embodiments, the aforementioned peptides are provided in connection with any adaptor protein which ensures uptake into virus-infectable cells and/or as a galenic formulation comprising peptides together with a compatible carrier.

The influenza preventive/therapeutic agent according to the present invention comprises at least one peptide of any one of the aforementioned peptides and/or at least one influenza virus replication inhibitor of any one of the aforementioned inhibitors as an active ingredient. This influenza preventive/therapeutic agent is effective against infections of both an influenza virus type A and type B.

Expression vectors comprising the polynucleotides encoding for the peptides described above have been introduced in to cells to enable them to secrete the peptides according to the present invention.

Influenza therapeutic agents comprising an influenza virus replication-inhibiting peptide disclosed herein have been developed.

The DNA or polynucleotides according to the present invention encode any one of the aforementioned peptides and is constituted of DNA, RNA, genomic DNA or PNA.

The expression vector according to the present invention includes the aforementioned DNA. Further, the cells according to the present invention are introduced with the aforementioned expression vector and secrete any one of the aforementioned peptides.

The aforementioned peptides may be contained in liposomes. The peptides in said liposomes are alkylated according to a preferred embodiment.

Besides these medical uses as described above, the influenza virus replication-inhibiting peptides according to the present invention can also be used as tools for identifying antiviral drugs. The identification of novel peptides like PB1₁₋₂₅A_{T6Y} and their ability to inhibit growth of both FluA and FluB validates the polymerase subunit PA and PB1 interaction as a novel target for the development of antiviral drugs with small molecules or other compounds specifically blocking the PA-PB1 interaction and inhibiting growth of several FluA and FluB strains, acting as broad-spectrum anti-influenza drugs.

In addition, the present invention provides an Enzyme-Linked ImmunoSorbent Assay (ELISA) based screening assay, to identify small molecule lead compounds which are antivirally active against influenza A and B viruses. Since they are effective against both virus types, such compounds represent an attractive alternative to neuraminidase inhibitors. Therefore, the present invention represents a major step toward a sorely needed, near-universal medicament against influenza virus, and one which, due to its protein-protein interaction domain target, will likely be less susceptible to the emergence of drug-resistant strains for which influenza is well known. A Fluorescence Polarization (FP) Assay is also provided.

The ELISA was established to better analyze the binding properties of PB1 to PA. It confirmed the type-specific binding of FluA PA and FluB PA to PB1₁₋₂₅A and PB1₁₋₂₅B, respectively, as shown in FIG. 1b. To further characterize the effect of the individual aa, competitive ELISA experiments using PB 1₁₋₂₅A-derived peptides were performed (Table 2). Peptides lacking the aa constituting the 3₁₀-helix failed to compete for binding, which is in agreement with the structure of the PA/PB1 binding site. Furthermore, peptides containing single Ala or Asp substitutions within the 3₁₀-helical domain—except for T6A—lost their ability to bind FluA PA (Table 3). This may be due to an allosteric effect or a loss of hydrogen bond contacts.

It has been found that the crucial PA interaction domain of PB1 consists of a 3₁₀-helix formed by amino acids (aa) X₅ to X₁₁. This domain is highly conserved and type-specific among both influenza A and B viruses (FIG. 1a).

Additionally, FluB PB1 was able to bind to FluA PA when these 25 aa were exchanged with the FluA PB1 sequence (FIG. 2).

The IC₅₀ values of FluA- and FluB-derived peptides (15-mer) for the PA-PB1₁₋₂₅A interaction were determined, as well as for a set of FluA/FluB chimeras (Table 1). Wild type PB1₁₋₁₅A efficiently inhibited FluA but not FluB PA binding to the cognate peptides, while PB1₁₋₁₅B blocked FluB PA but not FluA PA binding. Some of the chimeric peptides lost the ability to bind to FluA PA (Table 1). Surprisingly, the peptide PB1₁₋₁₅A_{T6Y,L7F} not only competed for binding to FluA PA, but also to FluB PA, albeit with less affinity than PB1₁₋₁₅B. The introduction of a Tyr at position 6 alone (PB1₁₋₁₅A_{T6Y}, which is highly conserved in FluB, led to decreased binding of FluA PA compared to the double mutant, yet binding was still better than with wild type peptide, whereas binding to FluB PA increased for PB1₁₋₁₅A_{T6Y} compared to PB1₁₋₁₅A_{T6Y,L7F}.

The L7F substitution resulted in a substantial loss of inhibitory activity (Table 1), indicating that the favorable binding properties of PB1₁₋₁₅A_{T6Y,L7F} can be attributed to T6Y. Structural analysis suggests that the FluB-derived Tyr at position 6 fits into an unexploited hydrophobic pocket in FluA PA, enhancing the binding between PB1₁₋₁₅A_{T6Y} and FluA PA (FIG. 1c). While this hydrophobic interaction was augmented for FluA with the introduction of Phe and Trp at position 6, binding to FluB PA deteriorated indicating a slightly different mode of interaction between FluB PA and PB1.

Both, the increased hydrophobic interaction as well as the entropic effect of water displacement might explain the enhanced binding of PB_{1-T6Y} to PA.

The favorable dual-binding properties were retained in the larger peptide PB1₁₋₂₅A_{T6Y}, which also bound to the PA subunits of several FluA and FluB strains (FIG. 1b and FIG. 3), demonstrating an affinity for a wide range of influenza virus subtypes. Polymerase reconstitution assays revealed that these dual-binding properties translate into virus type-independent inhibition of polymerase activity. PB1₁₋₂₅A_{T6Y} fused to GFP interfered with viral polymerase activity of both FluA and FluB, while PB1₁₋₂₅A-GFP and PB1₁₋₂₅B-GFP only inhibited the activity of their cognate subtypes (FIG. 1d). An accordant finding is observed using a coimmunoprecipitation experiment with proteins containing these sequences (FIG. 4).

In order to improve the delivery of the therapeutic active molecules, i.e. the synthesized or isolated influenza virus replication-inhibiting peptides according to the present invention, cell-penetrating peptides were used. Said cell-penetrating peptides are for example protein transduction domains (PTD) or transactivator proteins from lentiviruses, also known as Tat proteins. It has been shown, that PB1₁₋₂₅A_{T6Y} fused to the cell-penetrating domain of HIV-Tat (PB1₁₋₂₅A_{T6Y}-Tat, Table 4), inhibited the growth of both, FluA and FluB but not of an unrelated virus (FIG. 1e). As expected from our biochemical characterization, PB1₁₋₂₅A_{T6Y}-Tat led to a two-fold increase in growth inhibition of A/WSN/33 (H1N1) and the highly pathogenic H5N1 strain A/Thailand/1(Kan-1)/2004 compared to the wild type PB1₁₋₂₅A-Tat.

The influenza preventive/therapeutic agent according to the present invention is broadly effective against influenza A and B. Advantageously, the formulation of the present invention can be prepared synthetically upon demand in very short time. In the case of threatening pandemics caused by local or regional outbreaks of e.g. avian flu in Asian states or the most recent case of swine flu in Mexico the demand for broad acting influenza preventive/therapeutic agents is apparent.

Other aspects and features of the present invention will become apparent to those ordinarily skilled in the art upon review of the following description of specific embodiments of the invention in conjunction with the accompanying figures.

The object, characteristics, and advantages of the present invention as well as the idea thereof will be apparent to those skilled in the art from the descriptions given herein. It is to be understood that the embodiments and specific examples of the invention described herein below are to be taken as preferred examples of the present invention. These descriptions are only for illustrative and explanatory purposes and are not intended to limit the invention to these embodiments or examples. It is further apparent to those skilled in the art that various changes and modifications may be made based on the descriptions given herein within the intent and scope of the present invention disclosed herein.

### BRIEF DESCRIPTION OF THE DRAWINGS

FIG. 1 shows binding and inhibitory activity of PB1 ₁₋₂₅A_{T6Y}.

FIG. 1a shows in the upper panel the alignment of the consensus sequence of the N-terminal 25 aa of FluA (SEQ ID NO: 4) and FluB PB1 (SEQ ID NO: 5). Middle and lower panels show the alignment of the N-terminal 25 aa of all available FluA and of FluB sequences derived from PB1 full length sequences.

FIG. 1b shows the binding of HA-tagged PA subunits from cell extracts to the immobilized peptides corresponding to different domains of FluA PB1 and FluB PB1 Upper panels: Western blot of the PA-containing cell extracts used.

FIG. 1c shows the structure of FluA PB 1₁₋₁₅ and FluA PB1_{1-15T6Y} bound to FluA PA.

FIG. 1d shows the polymerase inhibitory activity of PB1₁₋₂₅-derived GFP fusion proteins in FluA and FluB polymerase reconstitution assays.

FIG. 1e shows a plaque reduction assay using PB 1₁₋₂₅A-Tat; PB1₁₋₂₅A_{T6Y}-Tat; PX-Tat (control peptide) with FluA, FluB and VSV (vesicular stomatitis virus).

FIG. 2 shows virus type-specific interaction of PA with PB1.

FIG. 2a shows PB1 chimeras used in tests according to FIG. 2b.

FIG. 2b shows the results of transfections with expression plasmids coding for the indicated PB1 proteins and the C-terminally hexahistidine-tagged PA of FluA (FluA PA_{His}).

FIG. 3 shows dual-binding properties of the FluA/B peptide chimera PB 1₁₋₂₅A_{T6Y} in comparison to PB1₁₋₂₅A and PB1₁₋₂₅B

FIG. 4a shows GFP-PB1 fusion proteins used in tests according to FIG. 4b.

FIG. 4b shows imunoblots based on formation of PB 1₁₋₂₅-derived GFP fusion proteins and HA-tagged PA of FluA and FluB.

### DETAILED DESCRIPTION

Embodiments of the present invention will now be described, by way of example only, with reference to the above mentioned Figures and the enclosed Tables.

### Materials and Methods

### Virus Strains

For the infection experiments A/WSN/33 (H1N1) according to Ghanem et al. (2007) and A/Thailand/1(Kan-1)/2004 according to Chockephaibulkit et al. (2005), B/Yamagat/73 according to Norton (1987) and VSV (serotype Indiana) as described in Schwemmle (1995) were used.

### Plasmid Constructions

Plasmids pCA-Flag-GFP and pCA-PB1₁₋₂₅A-GFP, pCA-PB1-HA, the FluA minireplicon plasmids and the expression plasmids for the FluB minireplicon are described in Ghanem (2007), Mayer (2007) and Pleschka (1996). The FluB minigenome expression plasmid, pPolI-lucRT_B, was obtained by cloning the firefly luciferase ORF (inverse orientation) flanked by the non-coding region of the segment 8 of the B/Yamagata/73 into the SapI-digested plasmid pPolI-SapI-Rib according to Pleschka (1996). For the construction of pCA-PB1₁₋₂₅B-GFP, a linker containing the first 25 codons of PB1 (B/Yamagata/73) was cloned into the EcoRI/NotI sites of pCA-Flag-GFP plasmid, replacing the Flag-coding sequence with PB1₁₋₂₅B. Site directed mutagenesis was carried out with pCA-PB1₁₋₂₅A-GFP to create the plasmid pCA-PB1₁₋₂₅A_{T6Y}-GFP. The ORFs of PB1 (B/Yamagata/73) and PA (A/SC35M, A/Thailand/1(KAN-1)/04, A/Vietnam/1203/04, B/Yamagata/73, B/Lee/40) were PCR amplified with sense primers containing an NotI site (FluA strains) or a EcoRI site (FluB strains) upstream of the initiation codon and antisense primers with a deleted stop codon followed by an XmaI site, a coding sequence for an HA-tag and a XhoI site. The PCR products were cloned into a modified pCAGGsvector (Schneider, 2003) digested either with EcoRI/XhoI or NotI/XhoI, resulting in pCA-PB1-HA or pCA-PA-HA plasmids, coding for C-terminal tagged versions of the polymerase subunits. To obtain the pCA-PA_{A/SC35M}-His plasmid, pCA-PA_{A/SC35M}-HA was digested with XmaI/XhoI and the HA coding sequence was replaced by a 6×His-linker. The A/B-chimeric expression plasmids were obtained by assembly PCR using the pCAPB1-HA plasmids of SC35M and B/Yamagata/73 and by cloning the resulting PCR product in pCA-PB1_{B/Yamagata/73}-HA digested with EcoRI/EcoRV.

### Reconstitution of the Influenza Virus Polymerase Activity

HEK293T cells were transiently transfected with a plasmid mixture containing either FluA- or FluB-derived PB1-, PB2-, PA- and NP-expression plasmids, polymerase I (Pol I)-driven plasmid transcribing an influenza A or influenza B virus-like RNA coding for the reporter protein firefly luciferase to monitor viral polymerase activity and with expression plasmids coding for the indicated GFP fusion proteins. Both minigenome RNAs were flanked by non-coding sequences of segment 8 of FluA and FluB, respectively. The transfection mixture also contained a plasmid constitutively expressing Renilla luciferase, which served to normalize variation in transfection efficiency. The reporter activity was determined 24 h post transfection and normalized using the Dual-Glu® Lufierase Assay System (Promega). The activity observed with transfection reactions containing Flag-GFP were set to 100%.

### Peptide Synthesis

The solid-phase synthesis of the peptides was carried out on a Pioneer automatic peptide synthesizer (Applied Biosystems, Foster City, USA) employing Fmoc chemistry with TBTU/diisopropylethyl amine activation. Side chain protections were as follows: Asp, Glu, Ser, Thr and Tyr: t-Bu; Asn, Gln and His: Trt; Arg: Pbf; Lys and Trp: Boc. Coupling time was 1 h. Double couplings were carried out if a difficult coupling was expected according to the program Peptide Companion (CoshiSoft/PeptiSearch, Tucson, USA). All peptides were generated as carboxyl amides by synthesis on Rapp S RAM resin (Rapp Polymere, Tübingen, Germany). Biotin was incorporated at the C-terminus of indicated peptides with Fmoc-Lys(Biotin)-OH (NovaBiochem/Merck, Nottingham, UK) and TBTU/diisopropylethylamine activation for 18 h, followed by coupling of Fmoc-B-Ala-OH for 1 h. Peptides were cleaved from the resin and deprotected by a 3 h treatment with TFA containing 3% triisobutylsilane and 2% water (10 ml/g resin). After precipitation with t-butylmethylether, the resulting crude peptides were purified by preparative HPLC (RP-18) with water/acetonitrile gradients containing 0.1% TFA and characterized by analytical HPLC and MALDI-MS. Some peptides were synthesized by peptides & elephants (Nuthetal, Germany) and subsequently purified and characterized as described above.

### Immunoprecipitation Experiments

HEK293T cells were transfected with the indicated plasmids in 6-well plates using Metafectene (Biontex, Martinsried, Germany). Cells were incubated 24 h post transfection with lysis buffer (20 mM Tris pH7.5, 100 mM NaCl, 0.5 mM EDTA, 0.5% NP-40, 1% Protease inhibitor Mix G, (Serva, Heidelberg, Germany), 1 mM DTT) for 15 min on ice. After centrifugation by 13.000 rpm at 4° C. supernatant was incubated with anti HA-specific antibodies coupled to agarose beads (Sigma) for 1 h at 4° C. After three washes with 1 ml of washing buffer (lysis buffer without protease inhibitor mix), bound material was eluted under denaturing conditions and separated on SDSPAGE gels and transferred to PVDF membranes. Viral polymerase subunits and GFP fusion proteins were detected with antibodies directed against the HA- (Covance, Berkeley, California) or His-(Qiagen) or GFP-tag (Santa Cruz Biotechnology).

### Plaque Reduction Assay

The experiments were carried out as described by Schmidke (2001) with modifications. Confluent MDCK cells were infected with 100 PFU of A/WSN/33, B/Yamagata/73, A/KAN-1, or VSV/Indiana in PBS containing BSA at room temperature. After removal of the inoculums, cells were overlaid with medium (DMEM with 20 mM Hepes, 0.01% DEAE Dextran, 0.001% NaHCO₃) containing 1% Oxoidagar and peptides at the indicated concentrations. After incubation for 24 h (VSV), 48 h (A/WSN/33, A/KAN-1) at 37° C. with 5% CO2, or 72 h at 33° C. with 5% CO₂ (B/Yamagata/73) respectively, cells were fixed with formaldehyde and stained with crystal violet. Plaques were counted and mean plaque number of the water control was set to 100%.

### ELISA

Microwell plates (Pierce) were incubated with saturating concentrations of peptides at room temperature, washed and subsequently incubated at room temperature with HA-tagged PA. To obtain PA-HA, 293t cells were seeded into 94 mm-dishes, transfected with the respective plasmid and treated with lysis buffer 24 h post transfection as described in detail by Meyer et al. (2007). After washing the microwell plates, the wells were incubated with an HA-specific primary antibody (Covance), followed by three washes and an incubation with a peroxidase-coupled secondary antibody (Jackson Immuno Research, Newmarket, UK) for further 30 min.

After the final wash step, ABTS-substrate (Sigma, ready-to-use solution) was added and the optical density was determined at 405 nm.

The competition ELISA was carried out as described above with the exception that the competitor peptides were added to wells of the plate with bound peptides prior to addition of the cell extract containing HA-tagged PA subunits.

### Fluorescence Polarization (FP) Assay

The test sample includes a known binding pair of proteins or protein subunits including a fluorescent label, which can be analyzed according to a preferred embodiment of the present invention by fluorescence polarization. Here, we use the interaction of Influenza A virus polymerase subunit PB1, represented by the first 25, N-terminal amino acids, and subunit PA. The test sample is then contacted with a candidate inhibitor compound and the fluorescence polarization is determined. The ability of the compound to cause dissociation of or otherwise interfere with or prevent binding of the proteins or protein subunits is monitored by fluorescence polarization (FP). FP measurements allow for discrimination between fluorescently labeled bound and unbound proteins, peptides, subunits or fragments thereof. The FP of the fluorescently labeled first fragment rotates rapidly in solution and, therefore, has randomized photo-selected distributions, which result in the small observed FP. When the fluorescently labeled first fragment of the first subunit interacts with the fragment of the second subunit, which is typically a larger, more slowly rotating molecule, the rotation of the fluorescently labeled first fragment slows and the fluorescence polarization increases. Accordingly, disruption of the subunit interaction by a test compound provides a decrease in the fluorescence polarization, which is indicative of inhibition of the protein interactions. The FP measurements in the presence of a test compound can be compared with the FP measurements in the absence of the test compound. Comparison can be made manually by the operator or automatically by a computer, especially in high throughput assays using 384-well plates.

For protein purification influenza A virus polymerase subunit PA was cloned into a suitable expression vector with a C-terminally attached 6×His-linker or hemagglutinine epitope (HA). Human 293T cells were transfected with the plasmid. Cell lysates were prepared 24 hours post transfection using lysis buffer (20 mM TrisHCl pH 7.5, 100 mM NaCl, 0.5 mM EDTA, 0.5% NP-40, 1 mM DTT and 1% Protase inhibitor mix) For purification from the lysate, PA subunit was bound to Ni- or anti-HA-agarose and washed with lysis buffer without protease mix. After elution with HA-peptide in 20 mM TrisHCl pH 7.5, 150 mM NaCl, 0.5 mM EDTA, 1 mM DTT and 5% Glycerol, PA-protein was concentrated when necessary using Vivaspin20 50K columns and frozen at −80° C. until further use. After thawing, the elution buffer was exchanged to low fluorescent grade reagents and any HA-peptide was removed simultaneously using 10-DG Bio-Gel columns.

Fluorescently labeled peptide corresponding to the 25 first N-terminal amino acids of Influenza A virus polymerase subunit PB1 at 3 nM concentration was added to 10 mikroM HA-PA in 20 mM TrisHCl pH 7.5, 150 mM NaCl, 0.5 mM EDTA, 1 mM DTT, 5% Glycerol and 100 mg/ml bovine gamma globulin. The mix was distributed into black 384-well plates to a total volume of 20 mikroliter per well and kept on ice. Test compounds solved in DMSO were added to a final concentration of 25 mikroM. After incubation for 10 minutes at room temperature, plates were read using an Infinite F200 reader (Tecan). FP values of the wells containing test compounds were compared to wells without test compounds, without DMSO and with peptide only.

Sequence alignment: Alignments were performed with MUSCLE as described in Edgar (2004) using the full-length sequences provided from the public influenza virus database.

Modelling: Manual docking of the mutated peptide into the PA(C)-PB1(N) crystal structure (He et al., 2008) and subsequent minimization was performed with Accelrys Discovery Studio.

### DETAILED DESCRIPTION OF THE TABLES AND FIGURES

Table 1a shows the inhibitory concentrations of FluA/FluB-derived peptides determined by competitive ELISA. Competitor peptides (0.048 to 3000 nM) were mixed with cell extracts containing HA-tagged PA from either FluA or FluB. Table 1 lists 12 competitive peptides. The first peptide PB1₁₋₁₅A is the FluA wild type; the second row shows the FluB wild type. For the peptides of rows 3 to 8 letters indicate FluB specific amino acids. Rows 9 to 12 list further competitive peptides with amino acids at position 6 being neither FluA nor FluB specific. S.D. is indicated in parenthesis. Asterisks indicate highest concentrations of peptides used without reaching 50% inhibition. Further competitive peptides which are not listed in the table but have effectively reached 50% inhibition at low peptide concentrations are PB1₁₋₁₅A_{T6I}A, PB1₁₋₁₅A_{T6L} and PB1₁₋₁₅A_{T6V}. Peptides with slightly lower inhibition activity are PB1₁₋₁₅A_{T6A} and PB 1₁₋₁₅A_{T6M} which are also not shown in Table 1a.

TABLE 1a
inhibitory concentrations of F1uA/F1uB-derived peptides determined
by competitive ELISA

| | | SEQ | | |
|---|---|---|---|---|
| | | ID | IC₅₀(nM) | |
| Competitive peptide | | NO: | PA (FluA) | PA (FluB) |
| PB1₁₋₁₅A | MDVNPTLLFLKVPAQ | 3 | 43.32 (+/−5.31) | >3000* |
| PB1₁₋₁₅B | -NI--YF--ID--I- | 6 | >3000* | 45.0 (+/−12.5) |
| PB1₁₋₁₅A_{D2N,V3I,L14I} | -NI----------I- | 7 | 6.69 (+/−1.73) | >3000* |
| PB1₁₋₁₅A_{L10I,K11D} | ---------ID---- | 8 | >3000* | >3000* |
| PB1₁₋₁₅A_{D2N,V3I} | -NI------------ | 9 | 12.96 (+/−3.98) | >3000* |
| PB1₁₋₁₅A_{T6Y,L7F} | -----YF-------- | 10 | 7.51 (+/−0.71) | 345.0 (+/−81.5) |
| PB1₁₋₁₅A_{L7F} | ------F-------- | 11 | >3000* | >3000* |
| PB1₁₋₁₅A_{T6Y} | -----Y--------- | 12 | 21.64 (+/−1.48) | 107.1 (+/−31.3) |
| PB1₁₋₁₅A_{T6F} | -----F--------- | 13 | 2.84 (+/−0.48) | 750.4 (+/−249.6) |
| PB1₁₋₁₅A_{T6W} | -----W--------- | 14 | 3.40 (+/−0.51) | 628.3 (+/−389.1) |
| PB1₁₋₁₅A_{T6H} | -----H--------- | 15 | 292.16 (+/−34.04) | >3000* |
| PB1₁₋₁₅A_{T6C} | -----C--------- | 16 | 43.58 (=/−5.67) | >3000* |

*highest concentration of competitive peptide used

A comprehensive and qualitative overview on further peptides with high inhibitory activity is provided in Table 1b. In the table the amino acid sequences at positions X₅ to X₁₀ of wild type A mutants are indicated.

TABLE 1b
qualitative overview of further preferred peptides

| AA | | | | | | |
|---|---|---|---|---|---|---|
| Position | X₅ | X₆ | X₇ | X₈ | X₉ | X₁₀ |
| wild type A | P | T | L | L | F | L |
| | | I | | I | Y | |
| | | L | | | W | |
| | | V | | | | |

In Table 1c the amino acid sequences at amino acid residues X₅ to X₁₀ of wild type A mutants are indicated. Said peptides exhibit lower activities than the above mentioned peptides according to Tables 1a and 1b.

TABLE 1c
qualitative overview of further peptides

| AA | | | | | | |
|---|---|---|---|---|---|---|
| Position | X₅ | X₆ | X₇ | X₈ | X₉ | X₁₀ |
| wild type A | P | T | L | L | F | L |
| | | M | | F | H | I |
| | | A | | M | L | |
| | | | | | R | |
| | | | | | S | |

Based on the above presented information and results, it is clear for the person skilled in the art, that the synthesized or isolated influenza virus replication-inhibiting peptides according to the invention comprise an amino acid sequence of X₅X₆X₇X₈X₉X₁₀, wherein X₅ is P; X₆ is T, Y, F, W, H, C, I, L, V, A or M; X₇ is L or F; X₈ is L, I, F or M; X₉ is F, Y, W, H, L, R or S, and X₁₀ is L, I or Y (SEQ ID NO: 17). Said amino acid sequence is at least 60%, preferably at least 70%, more preferably at least 80% or 90% identical to the polypeptide according to the wild type PB1₁₋₁₁A which is MDVNPTLLFLK (SEQ ID NO: 1).

Within the aforementioned group of peptides, those peptides are preferred which comprising the amino acid sequence of X₆X₇X₈X₉X₁₀, wherein X₆ is T, Y, F, W, H, C, I, L or V; X₇ is L or F; X₈ is L or I; X₉ is F, Y or W and X₁₀ is L (SEQ ID NO: 18). Even more preferred according to certain embodiments are peptides that comprise the amino acid sequence of X₆X₇, wherein X₆ is T, Y, F, W, H, C, I, L or V and X₇ is L or F (SEQ ID NO: 19).

Peptides according to the present invention comprise at least 11 residues X₁₋₁₁ according to preferred embodiments, whereby preferably the proteins comprise the amino acid sequence MDVNPX6X7LFLKVPAQ wherein X6 is selected from the group: T, Y, F, W, H. C, A, I, L, V or M and X7 is selected from the group L or F (SEQ ID NO: 20). A preferred peptide comprises an amino acid sequence elected from the group: MDVNPYFLFLKVPAQ (SEQ ID NO: 10), MDVNPYLLFLKVPAQ (SEQ ID NO: 12), MDVNPWLLFLKVPAQ (SEQ ID NO: 14) or MDVNPFLLFLKVPAQ (SEQ ID NO: 13).

According to further preferred embodiments of the present invention the peptides comprise at least 15 residues X₁₋₁₅ according to the wild type PB1₁₋₁₅A but not the wild type sequence MDVNPTLLFLKVPAQ (SEQ ID NO: 3).

Table 2 shows the 50%-inhibitory concentrations (IC₅₀) of FluA-derived PB1 peptides determined by competitive ELISA. Peptide PB1₁₋₂₅A was immobilized on microwell plates and incubated with increasing concentrations of competitor peptides and cell extract containing HA-tagged PA of FluA. Bound PA was detected by HA-specific antibodies as described above. S.D. is shown in parenthesis. Asterisks indicate highest concentrations of peptides used without detectable inhibitory effect. Grey boxes highlight amino acids that are part of the 3₁₀-helix, which comprises the core PA-binding region of PB1. Amino acids known to form hydrogen bonds with PA residues are represented in bold. The systematic truncation of the 25 mer peptide comprising the PA-binding domain of PB1 at the N- and C-terminus showed—based on the ELISA assay results—that i) the 25 mer peptide can be truncated at the C-terminus until the first 14 or even 13 N-terminal amino acids remain without losing ability to inhibit the bound peptide-PA interaction. Truncation at the C-terminus down to the first 12 or even 11 amino acids resulted in peptides which still showed considerable activity. The systematic truncation showed further that ii) N-terminal truncation is not possible without major loss in inhibitory activity of the peptide.

TABLE 2
Inhibitory concentration (IC₅₀) of FluA-derived PB1 peptides

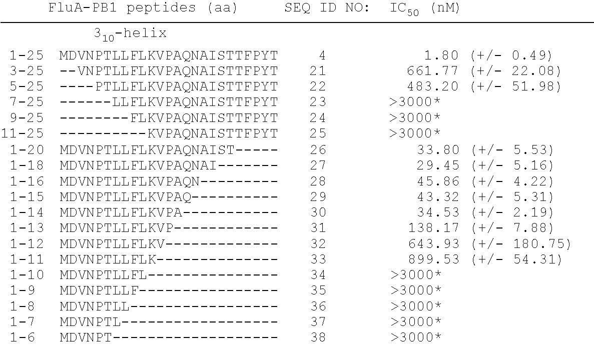

*highest concentration of competitive peptide used

Table 3 illustrates the inhibitory concentrations (IC₅₀) of FluA-derived competitor peptides determined by ELISA. Peptide PB1₁₋₂₅A was again immobilized on microwell plates and incubated with increasing concentrations of competitor peptide and cell extract containing HA-tagged PA of FluA. HA-specific antibodies detected bound PA. S.D. are shown in parenthesis. Asterisks indicate highest concentrations of peptides used without detectable inhibitory effect.

TABLE 3
Inhibitory concentrations (IC₅₀) of F1uA-derived PB1 peptides

| | | SEQ | |
|---|---|---|---|
| | | ID | |
| Competitive peptide | | NO: | IC50 in nM |
| PB1₁₋₁₅A | MDVNPALLFLKVPAQ | 3 | 43.32 (+/−5.31) |
| PB1₁₋₁₅A M1A | ADVNPTLLFLKVPAQ | 39 | 460.30 (+/−27.85) |
| PB1₁₋₁₅A D2A | MAVNPTLLFLKVPAQ | 40 | 209.17 (+/−44.62) |
| PB1₁₋₁₅A V3A | MDANPTLLFLKVPAQ | 41 | 154.93 (+/−18.18) |
| PB1₁₋₁₅A N4A | MDVAPTLLFLKVPAQ | 42 | >3000* |
| PB1₁₋₁₅A P5A | MDVNATLLFLKVPAQ | 43 | 2728.67 (+/−133.43) |
| PB1₁₋₁₅A T6A | MDVNPALLFLKVPAQ | 44 | 701.87 (+/−20.59) |
| PB1₁₋₁₅A L7A | MDVNPTALFLKVPAQ | 45 | >3000* |
| PB1₁₋₁₅A L8A | MDVNPTLAFLKVPAQ | 46 | >3000* |
| PB1₁₋₁₅A F9A | MDVNPTLLALKVPAQ | 47 | >3000* |
| PB1₁₋₁₅A L10A | MDVNPTLLFAKVPAQ | 48 | >3000* |
| PB1₁₋₁₅A K11A | MDVNPTLLFLAVPAQ | 49 | 1290.33 (+/−210.37) |
| PB1₁₋₁₅A V12A | MDVNPALLFLKAPAQ | 50 | 707.87 (+/−168.54) |
| PB1₁₋₁₅A P13A | MDVNPTLLFLKVAAQ | 51 | 257.93 (+/−36.76) |
| PB1₁₋₁₅A M1D | DDVNPTLLFLKVPAQ | 52 | 1375.67 (+/−268.11) |
| PB1₁₋₁₅A V3D | MDDNPTLLFLKVPAQ | 53 | >3000* |
| PB1₁₋₁₅A N4D | MDVDPTLLFLKVPAQ | 54 | >3000* |
| PB1₁₋₁₅A P5D | MDVNDTLLFLKVPAQ | 55 | >3000* |
| PB1₁₋₁₅A T6D | MDVNPDLLFLKVPAQ | 56 | 2067.67 (+/−584.98) |
| PB1₁₋₁₅A L7D | MDVNPTDLFLKVPAQ | 57 | >3000* |
| PB1₁₋₁₅A L8D | MDVNPTLDFLKVPAQ | 58 | >3000* |
| PB1₁₋₁₅A F9D | MDVNPTLLDLKVPAQ | 59 | >3000* |
| PB1₁₋₁₅A L10D | MDVNPTLLFDKVPAQ | 60 | >3000* |
| PB1₁₋₁₅A K11D | MDVNPTLLFLDVPAQ | 61 | >3000* |
| PB1₁₋₁₅A V12D | MDVNPTLLFLKDPAQ | 62 | 2302.67 (+/−280.39) |
| PB1₁₋₁₅A P13D | MDVNPTLLFLKVDAQ | 63 | 1097.47 (+/−217.54) |

*highest concentration of competitive peptide used

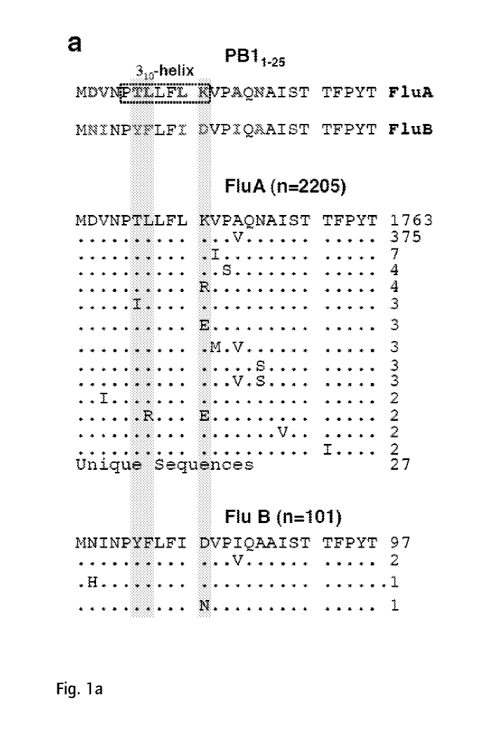

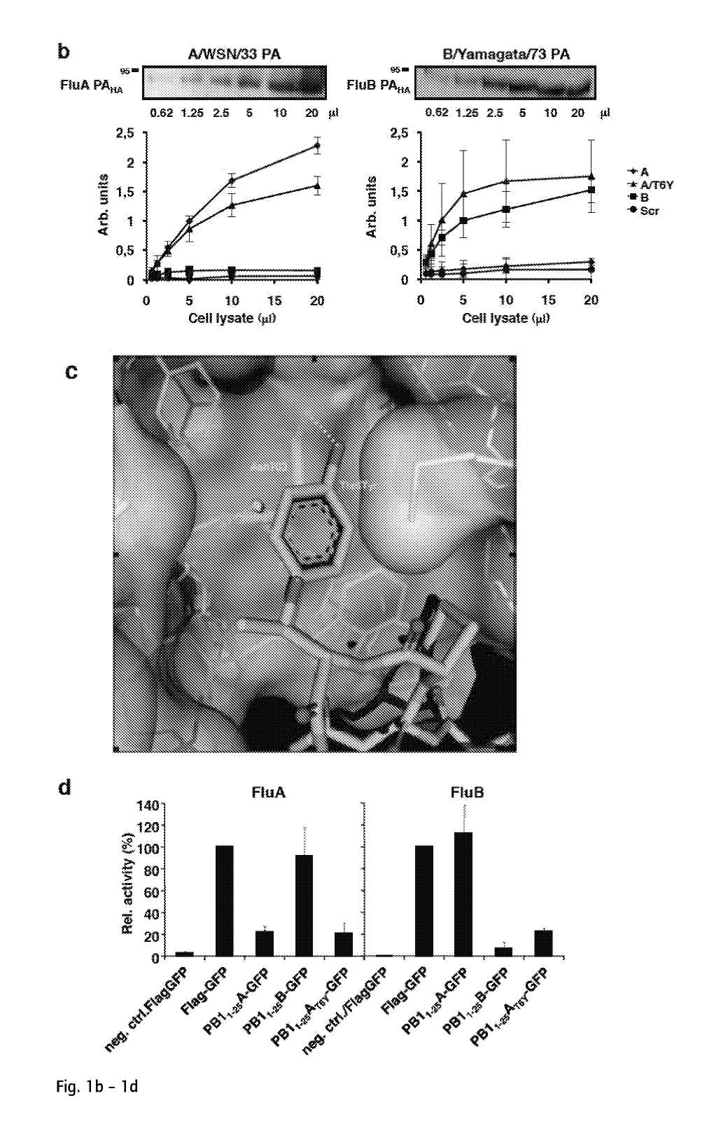

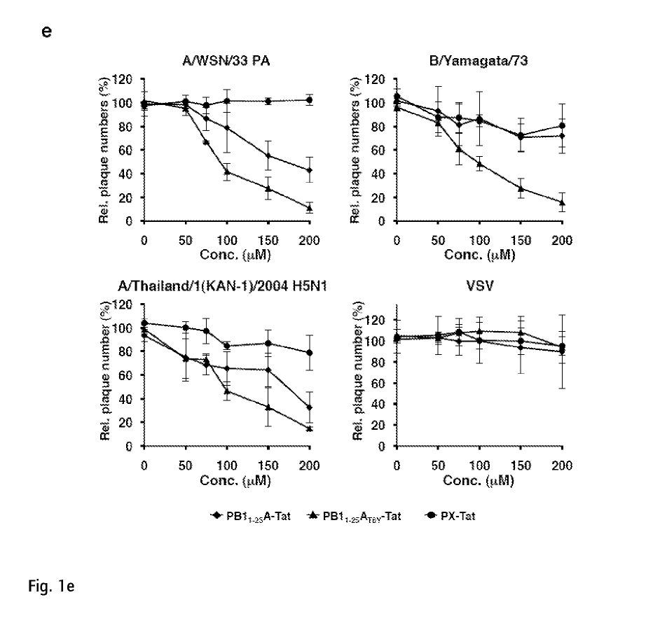

Based on FIG. 1 the binding and inhibitory activity of peptides according to the invention with a focus on the preferred protein PB1₁₋₂₅A_{T6Y} shall be illustrated in the following part of the description. FIG. 1a shows in the upper panel the alignment of the consensus sequence of the N-terminal 25 aa of FluA and FluB PB1 (SEQ ID NO: 4 and 5, respectively), wherein the dotted box indicates the 3₁₀-helix comprising the core PA-binding domain of PB1 and the FluA-specific and FluB-specific aa are printed in bold letters. Middle and lower panels show the alignment of the N-terminal 25 aa of all available FluA and FluB sequences derived from PB1 full length sequences provided by the NCBI influenza virus database. The binding of HA-tagged PA subunits from cell extracts to the immobilized peptides corresponding to the domains of FluA PB1 (PB 1₁₋₂₅A), FluB PB1 (PB 1₁₋₂₅B) or FluA PB1 T6Y (PB1₁₋₂₅A_{T6Y}) determined by ELISA is shown in FIG. 1b. Signals using the cognate peptide and lysate were normalized to 1. Binding of the PA subunits to the control peptides was not observed. Upper panels: Western blot of the PA-containing cell extracts used. Molecular weights shown in kilodaltons.

FIG. 1c provides some graphic information on the structure of FluA PB1₁₋₁₅ bound to FluA PA. T6 forms a hydrogen bond to a water molecule. Molecular modeling suggests that the aromatic side chain in the mutant T6Y fits into a hydrophobic pocket and displaces the water molecule.

The polymerase inhibitory activity of PB 1₁₋₂₅-derived GFP fusion proteins in FluA and FluB polymerase reconstitution assays is shown in FIG. 1d. The activity in experiments containing all viral plasmids and Flag-GFP was set to 100%.

FIG. 1e shows a plaque reduction assay using PB1₁₋₂₅A-Tat; PB1₁₋₂₅A_{T6Y}-Tat; PX-Tat (control peptide) with FluA, FluB and VSV (vesicular stomatitis virus). A H₂O control was used to standardize the assay to 100%. Note that PB1₁₋₂₅B-Tat could not be tested due to insolubility. Error bars represent S.D.

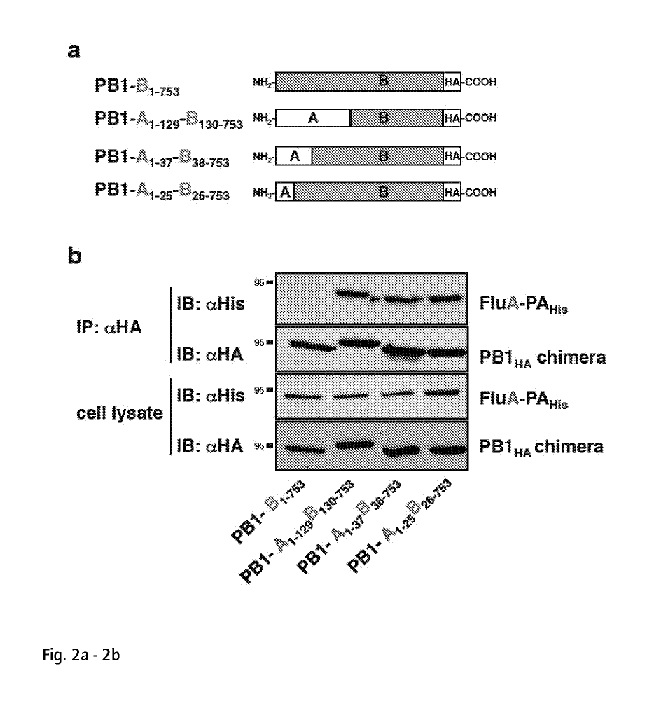

Virus type-specific interaction of PA with PB1 is illustrated in FIG. 2. FIG. 2a shows A/SC35M- and B/Yamagata/73-derived PB1 chimeras used in tests according to FIG. 2b. Note that all PB1 proteins were expressed with C-terminal HA-tags. FIG. 2b shows human 293T cells which were transfected with expression plasmids coding for the indicated PB1 proteins and the C-terminally hexahistidine-tagged PA of FluA (FluAPA_{His}). Cell lysates were prepared 24 hours post transfection and subjected to immunoprecipitation (IP) using anti-HA (aHA) agarose. Precipitated material was separated by SDS-PAGE and analyzed by Western blot for the presence of either His- or HA-tagged polymerase subunits using appropriate antibodies. Protein expression was controlled by analyzing equal amounts of cell lysate. Molecular weights are shown in kilodaltons. The 25-mer peptide, PB1₁₋₂₅A, comprising a helical domain inhibits the polymerase activity and replication of FluA, whereas the activity of FluB polymerase is not affected.

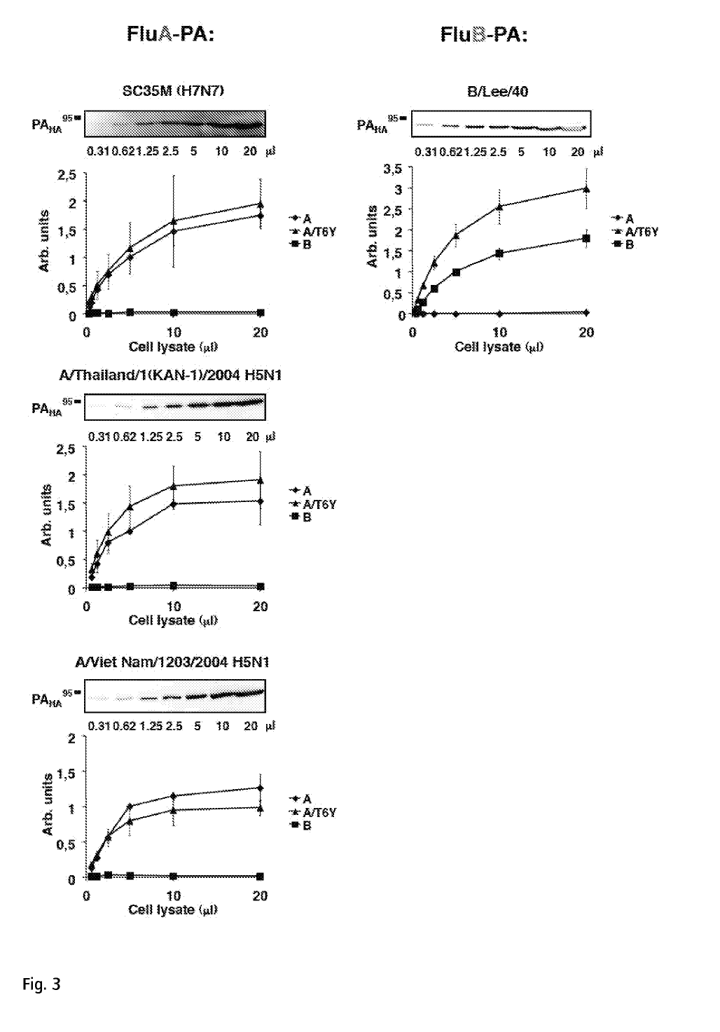

In FIG. 3 dual-binding properties of the FluA/B peptide chimera PB1₁₋₂₅A_{T6Y} are illustrated. The Lower panels show peptides PB1₁₋₂₅A, PB1₁₋₂₅B or PB1₁₋₂₅A_{T6Y} immobilized on microwell plates and incubated with increasing concentrations of cell extract containing the indicated PA-HA from FluA or FluB strains. Bound PA-HA was detected by HA-specific antibodies and peroxidase-labeled secondary antibodies. Binding efficiency was quantified by measuring substrate conversion at 405 nm. S.D. are indicated by error bars. Experiments were repeated in triplicates. Upper panels show analysis of corresponding amounts of cell lysate by Western blot controlled protein expression. Molecular weights are shown in kilo-daltons.

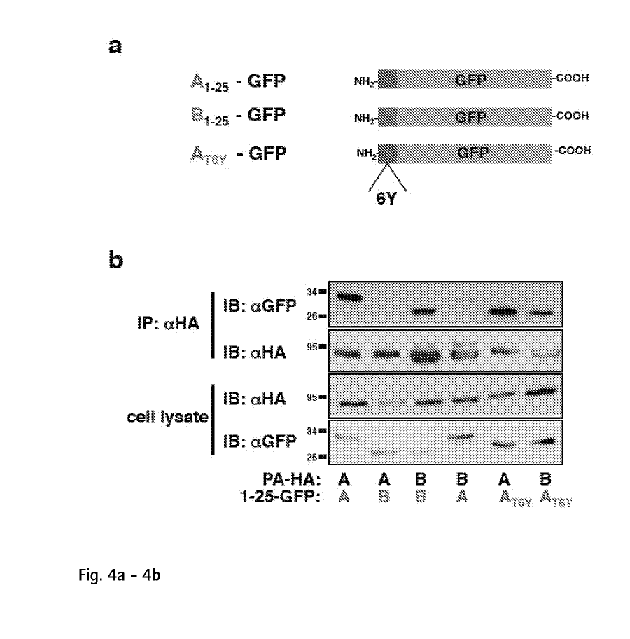

FIG. 4a shows GFP-PB1 fusion proteins used in tests according to FIG. 4b. The complex formation of PB1₁₋₂₅-derived GFP fusion proteins and HA-tagged PA of FluA and FluB is shown in FIG. 4b. Indicated proteins were expressed in human 293T cells and binding of the GFP fusion proteins was analyzed by immunoprecitation (IP) of PA using anti-HA agarose and subsequent immunoblotting (IB). Precipitated material was analyzed by Western blot using the indicated antibodies for the presence of either HA-tagged PA or GFP. Molecular weights are shown in kilodaltons.

The above-described embodiments of the present invention are intended to be examples only. Alterations, modifications and variations may be effected to the particular embodiments by those of skill in the art without departing from the scope of the invention, which is defined solely by the claims appended hereto. All references are herein incorporated by reference.

### REFERENCES

1. http://www.who.int/csr/disease/influenza/vaccinerecommendations1/en/.

2. Moscona, A. N Engl J Med 353, 1363-1373 (2005).

3. Davies, W. L. et al. Science 144, 862-863 (1964).

4. Moscona, A. N Engl J Med 360, 953-956 (2009).

5. Dharan, N. J. et al. JAMA 301, 1034-1041 (2009).

6. Pilger, B. D., Cui, C. & Coen, D. M. Chem Biol 11, 647-654 (2004).

7. Brownlee, G. G. & Sharps, J. L. J Virol 76, 7103-7113 (2002).

8. Perales, B. & Ortin, J. J Virol 71, 1381-1385 (1997).

9. Fodor, E. et al. J Virol 76, 8989-9001 (2002).

10. He, X. et al. Nature 454, 1123-1126 (2008).

11. Obayashi, E. et al. Nature 454, 1127-1131 (2008).

12. Ghanem, A. et al. J Virol 81, 7801-7804 (2007).

13. Dostmann, W. R. et al. Proc Natl Acad Sci U S A 97, 14772-14777 (2000).

14. Chokephaibulkit, K. et al. Pediatr Infect Dis J 24, 162-166 (2005).

15. Norton, G. P. et al. Virology 156, 204-213 (1987).

16. Schwemmle, M., Weining, K. C., Richter, M. F., Schumacher, B. & Staeheli, P. Virology 206, 545-554 (1995).

17. Mayer, D. et al. J Proteome Res 6, 672-682 (2007).

18. Pleschka, S. et al. J Virol 70, 4188-4192 (1996).

19. Schneider, U., Naegele, M., Staeheli, P. & Schwemmle, M. J Virol 77, 11781-11789 (2003).

20. Schmidtke, M., Schnittler, U., Jahn, B., Dahse, H. & Stelzner, A. J Virol Methods 95, 133-143 (2001).

21. Edgar, R. C. Nucleic Acids Res 32, 1792-1797 (2004).

1. A synthesized or isolated influenza. virus replication-inhibiting peptide that competitively inhibits protein-protein interaction of the PA and PB1 subunits Of the heterotrimeric viral RNA polymerase complex of both influenza Virus Types A and B, comprising an amino acid sequence, comprising the sequence of X₅X₆X₇X₈X₉ X₁₀, wherein X₅ is P; X₆ is T, Y, F, W, H, C, I, L, V, A or M; X₇ is L or F; X₈ is L, F M; X₉ is F, Y, W, H, L, R or S; and X₁₀ is L, I or Y (SEQ LD NO: 2), said amino acid sequence being at least 60%, 70%, 80% or 90% identical to the polypeptide according to the wild type PB1₁₋₁₁A which is MDVNPTLLFLK (SEQ ID NO: 1), wherein said wild type PB1₁₋₁₁A is excluded.

2. A peptide according to claim 1 comprising the amino acid sequence being at least 66%, 73%, 79%, 86% or 93% identical to the polypeptide according to the wild type PB1₁₋₁₅A which is MDVNPTLLFLKVPAQ (SEQ ID NO: 3). wherein said wild type PB1₁₋₁₅A is excluded.

3. A peptide according to claim 1 comprising the amino acid sequence of X₆X₇X₈X₉X₁₀, wherein X₆ is T, Y, W, H, C, I, L or V; X₇ is L or F; X₈ is L or I; X₉ is F, Y or W and X₁₀ is L (SEQ ID NO: 18).

4. A peptide according to claim 3 comprising the amino acid sequence of X₆X₇, wherein X₆ is T, Y, F, C, I, L or V and X₇ is L or F (SEQ ID NO: 19).

5. A peptide according to claim 1 wherein the amino acid sequence comprises 15 residues X₁₋₁₅.

6. A peptide according to claim 1 comprising amino acid sequence MDVNPX₆X₇LFLKVPAQ wherein X₆ is selected from the group: T, Y, F, W, H, C, A, L, V or M and X₇ is selected from the group L or F (SEQ ID NO: 20).

7. A peptide according to claim 6 comprising an amino acid sequence selected from the group: MDVNPYFLFLKVPAQ (SEQ ID NO: 10), MDVNPYLLFLKVPAQ (SEQ ID NO: 12), MDVNPWLLFLKVPAQ (SEQ NO: 14) or MDVNPFLLFLKVPAQ (SEQ ID NO: 13).

8. An influenza virus replication inhibitor, comprising the peptide, of claim 1 fused to a cell-penetrating peptide, preferably a cell-penetrating domain of HIV-Tat, as an active ingredient.

9. The influenza virus replication inhibitor of claim 8, which inhibits replication of influenza A and influenza B strains.

10. An influenza preventive/therapeutic agent, comprising the peptide of claim 1 as an active ingredient.

11. The influenza preventive/therapeutic agent of claim 10, which is effective against influenza A and influenza B strains.

12. An isolated polynucleotide that encodes a peptide according to claim 1.

13. A galenic formulation comprising a peptide according to claim 1 and a compatible carrier.

14. A medicament comprising a peptide of claim 1.

15. A method for the treatment of influenza in a subject, comprising administering to the subject a peptide of claim 1.

16. (canceled)

17. A method for determining influenza polymerase subunit interaction inhibitors based on an ELISA Or a Fluorescence Polarisation Assay comprising the use of a peptide of claim 1 wherein said wild types are included, preferably as a competitive inhibitor.


SEQUENCE LISTING (63 sequences)

SEQ ID NO: 1 - protein, 11 aa, Artificial Sequence
  Made in a lab
MDVNPTLLFLK

SEQ ID NO: 2 - protein, 6 aa, Artificial Sequence
  Made in a lab
PXXXXX

SEQ ID NO: 3 - protein, 15 aa, Artificial Sequence
  Made in a lab
MDVNPTLLFLKVPAQ

SEQ ID NO: 4 - protein, 25 aa, Influenza A
MDVNPTLLFLKVPAQNAISTTFPYT

SEQ ID NO: 5 - protein, 25 aa, Influenza B
MNINPYFLFIDVPIQAAISTTFPYT

SEQ ID NO: 6 - protein, 15 aa, Artificial Sequence
  Made in a lab
MNINPYFLFIDVPIQ

SEQ ID NO: 7 - protein, 15 aa, Artificial Sequence
  Made in a lab
MNINPTLLFLKVPIQ

SEQ ID NO: 8 - protein, 15 aa, Artificial Sequence
  Made in a lab
MDVNPTLLFIDVPAQ

SEQ ID NO: 9 - protein, 15 aa, Artificial Sequence
  Made in a lab
MNINPTLLFLKVPAQ

SEQ ID NO: 10 - protein, 15 aa, Artificial Sequence
  Made in a lab
MDVNPYFLFLKVPAQ

SEQ ID NO: 11 - protein, 15 aa, Artificial Sequence
  Made in a lab
MDVNPTFLFLKVPAQ

SEQ ID NO: 12 - protein, 15 aa, Artificial Sequence
  Made in a lab
MDVNPYLLFLKVPAQ

SEQ ID NO: 13 - protein, 15 aa, Artificial Sequence
  Made in a lab
MDVNPFLLFLKVPAQ

SEQ ID NO: 14 - protein, 15 aa, Artificial Sequence
  Made in a lab
MDVNPWLLFLKVPAQ

SEQ ID NO: 15 - protein, 15 aa, Artificial Sequence
  Made in a lab
MDVNPHLLFLKVPAQ

SEQ ID NO: 16 - protein, 15 aa, Artificial Sequence
  Made in a lab
MDVNPCLLFLKVPAQ

SEQ ID NO: 17 - protein, 6 aa, Artificial Sequence
  Made in a lab
PXXXXX

SEQ ID NO: 18 - protein, 5 aa, Artificial Sequence
  Made in a lab
XXXXL

SEQ ID NO: 19 - protein, 2 aa, Artificial Sequence
  Made in a lab
XX

SEQ ID NO: 20 - protein, 15 aa, Artificial Sequence
  Made in a lab
MDVNPXXLFLKVPAQ

SEQ ID NO: 21 - protein, 23 aa, Artificial Sequence
  Made in a lab
VNPTLLFLKVPAQNAISTTFPYT

SEQ ID NO: 22 - protein, 21 aa, Artificial Sequence
  Made in a lab
PTLLFLKVPAQNAISTTFPYT

SEQ ID NO: 23 - protein, 19 aa, Artificial Sequence
  Made in a lab
LLFLKVPAQNAISTTFPYT

SEQ ID NO: 24 - protein, 17 aa, Artificial Sequence
  Made in a lab
FLKVPAQNAISTTFPYT

SEQ ID NO: 25 - protein, 15 aa, Artificial Sequence
  Made in a lab
KVPAQNAISTTFPYT

SEQ ID NO: 26 - protein, 20 aa, Artificial Sequence
  Made in a lab
MDVNPTLLFLKVPAQNAIST

SEQ ID NO: 27 - protein, 18 aa, Artificial Sequence
  Made in a lab
MDVNPTLLFLKVPAQNAI

SEQ ID NO: 28 - protein, 16 aa, Artificial Sequence
  Made in a lab
MDVNPTLLFLKVPAQN

SEQ ID NO: 29 - protein, 15 aa, Artificial Sequence
  Made in a lab
MDVNPTLLFLKVPAQ

SEQ ID NO: 30 - protein, 14 aa, Artificial Sequence
  Made in a lab
MDVNPTLLFLKVPA

SEQ ID NO: 31 - protein, 13 aa, Artificial Sequence
  Made in a lab
MDVNPTLLFLKVP

SEQ ID NO: 32 - protein, 12 aa, Artificial Sequence
  Made in a lab
MDVNPTLLFLKV

SEQ ID NO: 33 - protein, 11 aa, Artificial Sequence
  Made in a lab
MDVNPTLLFLK

SEQ ID NO: 34 - protein, 10 aa, Artificial Sequence
  Made in a lab
MDVNPTLLFL

SEQ ID NO: 35 - protein, 9 aa, Artificial Sequence
  Made in a lab
MDVNPTLLF

SEQ ID NO: 36 - protein, 8 aa, Artificial Sequence
  Made in a lab
MDVNPTLL

SEQ ID NO: 37 - protein, 7 aa, Artificial Sequence
  Made in a lab
MDVNPTL

SEQ ID NO: 38 - protein, 6 aa, Artificial Sequence
  Made in a lab
MDVNPT

SEQ ID NO: 39 - protein, 15 aa, Artificial Sequence
  Made in a lab
ADVNPTLLFLKVPAQ

SEQ ID NO: 40 - protein, 15 aa, Artificial Sequence
  Made in a lab
MAVNPTLLFLKVPAQ

SEQ ID NO: 41 - protein, 15 aa, Artificial Sequence
  Made in a lab
MDANPTLLFLKVPAQ

SEQ ID NO: 42 - protein, 15 aa, Artificial Sequence
  Made in a lab
MDVAPTLLFLKVPAQ

SEQ ID NO: 43 - protein, 15 aa, Artificial Sequence
  Made in a lab
MDVNATLLFLKVPAQ

SEQ ID NO: 44 - protein, 15 aa, Artificial Sequence
  Made in a lab
MDVNPALLFLKVPAQ

SEQ ID NO: 45 - protein, 15 aa, Artificial Sequence
  Made in a lab
MDVNPTALFLKVPAQ

SEQ ID NO: 46 - protein, 15 aa, Artificial Sequence
  Made in a lab
MDVNPTLAFLKVPAQ

SEQ ID NO: 47 - protein, 15 aa, Artificial Sequence
  Made in a lab
MDVNPTLLALKVPAQ

SEQ ID NO: 48 - protein, 15 aa, Artificial Sequence
  Made in a lab
MDVNPTLLFAKVPAQ

SEQ ID NO: 49 - protein, 15 aa, Artificial Sequence
  Made in a lab
MDVNPTLLFLAVPAQ

SEQ ID NO: 50 - protein, 15 aa, Artificial Sequence
  Made in a lab
MDVNPTLLFLKAPAQ

SEQ ID NO: 51 - protein, 15 aa, Artificial Sequence
  Made in a lab
MDVNPTLLFLKVAAQ

SEQ ID NO: 52 - protein, 15 aa, Artificial Sequence
  Made in a lab
DDVNPTLLFLKVPAQ

SEQ ID NO: 53 - protein, 15 aa, Artificial Sequence
  Made in a lab
MDDNPTLLFLKVPAQ

SEQ ID NO: 54 - protein, 15 aa, Artificial Sequence
  Made in a lab
MDVDPTLLFLKVPAQ

SEQ ID NO: 55 - protein, 15 aa, Artificial Sequence
  Made in a lab
MDVNDTLLFLKVPAQ

SEQ ID NO: 56 - protein, 15 aa, Artificial Sequence
  Made in a lab
MDVNPDLLFLKVPAQ

SEQ ID NO: 57 - protein, 15 aa, Artificial Sequence
  Made in a lab
MDVNPTDLFLKVPAQ

SEQ ID NO: 58 - protein, 15 aa, Artificial Sequence
  Made in a lab
MDVNPTLDFLKVPAQ

SEQ ID NO: 59 - protein, 15 aa, Artificial Sequence
  Made in a lab
MDVNPTLLDLKVPAQ

SEQ ID NO: 60 - protein, 15 aa, Artificial Sequence
  Made in a lab
MDVNPTLLFDKVPAQ

SEQ ID NO: 61 - protein, 15 aa, Artificial Sequence
  Made in a lab
MDVNPTLLFLDVPAQ

SEQ ID NO: 62 - protein, 15 aa, Artificial Sequence
  Made in a lab
MDVNPTLLFLKDPAQ

SEQ ID NO: 63 - protein, 15 aa, Artificial Sequence
  Made in a lab
MDVNPTLLFLKVDAQ